(19) United States
(12) Patent Application Publication
(10) Pub. No.: US 2006/0003346 A1
(43) Pub. Date: Jan. 5, 2006

(54) Method for treating hepatitis

(76) Inventors: Ashok Amin, Blacksburg, VA (US); Steven Abramson, Rye, NY (US)

Correspondence Address: BROWDY AND NEIMARK, P.L.L.C.;624 NINTH STREET, NW, SUITE 300, WASHINGTON, DC, 20001-5303, US

(21) Appl. No.: 11/071,886

(22) Filed: Mar. 4, 2005

Related U.S. Application Data
(63) Continuation-in-part of application No. 09/925,970, filed on Aug. 10, 2001, now Pat. No. 6,881,407.
(60) Provisional application No. 60/224,363, filed on Aug. 11, 2000.

(51) Int. Cl. C12Q 1/68; G01N 33/53; A61K 39/395; A61K 39/40; A61K 39/42; A61K 31/44; A01N 43/40

(52) U.S. Cl. 435/6; 435/7.1

(57) ABSTRACT

Hepatitis can be treated by administering to a patient in need thereof an effective amount of a compound that neutralizes the effects of secreted TNF-alpha.

### CROSS-REFERENCE TO RELATED APPLICATIONS

The present application is a continuation in part of application Ser. No. 09/925,970, filed Aug. 10, 2001, which claims priority from provisional application Ser. No. 60/224,363, filed Aug. 11, 2000, the entire contents of both of which are hereby incorporated by reference—.

### FIELD OF THE INVENTION

The present invention is directed to a method for treating hepatitis by inhibiting the production of TNF-alpha.

### BACKGROUND OF THE INVENTION

Tumor necrosis factor-alpha (TNA-alpha, also known as cachetin), is a mammalian protein capable of inducing a variety of effects on numerous cell types. TNF-alpha was initially characterized by its ability to cause lysis of tumor cells, and is produced by activated cells such as mononuclear phagocytes, T-cells, B-cells, mast cells and NK cells. Because the deleterious effects which can result from an over-production or an unregulated production of TNF-alpha are extremely serious, considerable efforts have been made to control or regulate serum levels of TNF.

The numerous biological effects of TNF-alpha and the closely related cytokine TNF-beta (lymphotoxin), are mediated by two transmembrane receptors, both of which have been cloned. The 55 receptor, also termed TNF-R55. TNF-RI, or TNFR-beta, is a 55 kd glycoprotein which has been shown to transduce signals, resulting in cytotoxic, anti-viral and proliferative activities of TNF-alpha.

The p75 receptor, also termed TNF-R75, TNF-RII, or TNF-alpha, is a 75 kd glycoprotein that has also been shown to transduce cytotoxic and proliferative signals as well as signals resulting in the secretion of GM-CSF. The extracellular domains of the two receptors are 28% identical in primary structure and have in common a set of four subdomains defined by numerous conserved cysteine residues. The p75 receptor differs, however, by having a region adjacent to the transmembrane domain that is rich in proline residues and contains sites of O-linked glycosylation. The cytoplasmic domains of the two receptors share no apparent homology, which is consistent with observations that they can transduce different signals to the interior of the cell.

There are a number of drugs that have been found to inhibit TNF-alpha activity, the best known of which are etanercept (ENBREL), which is based on a p75:pC receptor, and a chimeric monoclonal antibody that neutralizes the activity of TNF, such as infliximab (REMICADE). Etanercept is an extracellular ligand binding protein of the human p75 TNF receptor linked to the Fc portion of human IgG1. Infliximab is a humanized monoclonal antibody that neutralizes the activity of secreted TNF. Both etanercept and infliximab potently bind TNF and block inflammation by inhibiting the downstream effect of this cytokine.

Among other TNF-alpha inhibitors that can be used are adalimubab (HUMIRA®), and selected NSAIDS such as indomethacin, ibuprofen, and fenoprofen, which may act as PPAR-gamma agonists. Other TNF-alpha inhibitors include sulfated disaccharide molecules, such as those prepared by cleaving heparin with the enzyme heparinase I. Atrial natriuretic peptide (ANP) has also been found to inhibit TNF-alpha

Hepatitis is an inflammatory disorder which can be caused by viral infections, including Epstein-Barr, cytomegalovirus, and hepatitis A-E viruses. Hepatitis causes acute liver inflammation in the portal and lobular region, followed by fibrosis and tumor progression.

### SUMMARY OF THE INVENTIONS

It is an object of the present invention to overcome the aforesaid deficiencies in the prior art.

It is an object of the present invention to provide a treatment for hepatitis by inhibiting TNF-alpha.

It is another object of the present invention to treat hepatitis by administering to a patient suffering from hepatitis an effective amount of a compound that neutralizes the activity of secreted TNF-alpha.

It is a further object of the present invention to treat hepatitis A-E by administering to a patient suffering therefrom an effective amount of a compound that neutralizes the effect of secreted TNF by inhibiting p75:FC.

It is another object of the present invention to treat hepatitis by administering to a patient suffering hepatitis an effective amount of a compound that neutralizes the effect of secreted TNF which is a humanized monoclonal antibody.

According to the present invention, compounds that neutralize the effects of secreted TFN reverse the clinical symptoms associated with hepatitis. Thus, the present invention includes treating a patient suffering from hepatitis with a compound that neutralizes the effects of secreted TNF by inhibiting p75:FC.

### DETAILED DESCRIPTION OF THE INVENTION

Hepatitis A-E, as well as alcoholic hepatitis, can be treated by administering to a patient in need thereof an effective amount of a compound that neutralizes the activity of secreted TNF. One example of such a compound is a ligand binding protein of the human p75 TNF receptor linked to the Fc portion of human IgG1, or a chimeric or humanized monoclonal antibody that neutralizes the activity of TNF. These types of compounds have been found to reverse evidence of hepatic inflammation and viral load associated with active hepatitis.

TNF-alpha radiates its effect via two distinct high affinity TNF receptors (TNFR), p55 and p75. They are similar in their extracellular domains but have distinct intracellular domains and different distributions. TNFRs on the cell surface are cleaved by proteolytic enzymes and released into circulation. An increase of these serum soluble TNFRs (sTNFRs) is believed to reflect enhanced expression of these receptors on various cells, and increased activation of the TNF-alpha system. The role of sTNFR is still unclear. Some authors have suggested that the effect of sTNFRs may be concentration dependent. In vitro, at low concentrations, it may act to stabilize the activity of TNF-alpha, and at high concentrations it may act to inhibit TNF-alpha.

There have been numerous studies indicating that the cytokine system plays an important role in the pathogenesis of hepatitis C infection with respect to both severity and chronicity. TNF-alpha is a cytokine secreted mainly by activated macrophages and lymphocytes and is though to have a wide array of effects, including antineoplastic, antiviral, and immunomodulatory properties. The hepatitis C virus has been reported to induce TNF-alpha gene expression and TNF-alpha itself. In patients with chronic hepatitis C infection, cytotoxic T-lymphocytes are thought to play a major role in the production of TNF-alpha. In addition, TNF-alpha has been shown to induce hepatitis when injected into humans and rodents.

TNF-alpha may play a key role in the recruitment of monocytes, macrophages, lymphocytes, and the expression of adhesin molecules. It is thought to be pivotal in the inflammatory process, and may mediate apoptosis of hepatitis C infected hepatic cells as well as “bystander” cells. It is quite possible that the hepatitis C virus requires the TNF receptor as one of the co-receptors, as observed in other viral infections. The hepatitis virus has been reported to induce TNF-alpha-dependent apoptosis in hepatocytes while binding through viral core proteins. TNF-alpha and the hepatitis C core proteins together may be required to induce apoptosis in hepatocytes. Etanercept and other TNF-alpha inhibitors may inhibit co-signals generated by TNF-alpha via the death domain of its receptor for hepatic apoptosis and infection process. However, an article by Ray et al. in 1998 reported that hepatitis C core protein inhibits TNF-alpha induced apoptosis. Zhu N, Khoshnan A, Schneider R, Matsumoto M, Dennert G, Ware C, Lai M M. Hepatitis C virus core protein binds to the cytoplasmic domain of tumor necrosis factor (TNF) receptor 1 and enhances TNF-induced apoptosis. J Virol. (May 1998) 72(5):3691-7.

In this manner, TNF-alpha may be an important determinant of activity and chronicity of hepatitis infection. It should be noted that interferon alpha 2b, which is one of the therapeutic agents for hepatitis C, up regulates soluble TNF-alpha receptors following the first dose of recombinant interferon alpha 2b. In other studies the sustained responders to treatment with interferon alpha 2b showed a significant decrease in the levels of soluble TNFRp75 but not p55 compared to pretreatment levels.

Previous studies have shown that there is an up-regulation of TNF-alpha and TNF-alpha receptors p55 and p75 in patients with chronic hepatitis C infection (Kallinowski, 1997). The levels of TNFR appear to correlate with the severity of liver diseases in the patients (Kallinowski, 1997 and Itoh, 1999). Specifically, TNFRp75 has been reported to have significant correlations with serum levels of AST, ALT, GGT and gamma globulin in patients with chronic hepatitis C (Itoh, 1999).

Additionally, in support of the theory that anti-TNF therapy may have a role in the treatment of hepatitis C infection, Sakimoto et al. in 2000 reported in an animal model that pretreatment with anti-TNF monoclonal antibodies prevented liver injury in mice.

There has been a variable response of hepatitis C patients treated with etanercept, which is most likely due to a variety of viral and host factors, including polymorphisms of the TNF-alpha promoter gene at positions −238 and −308. Polymorphisms in the TNF-alpha gene have been reported to be associated with hepatitis C infection (Hohler, Kruger, 1998). TNF-alpha promoter gene polymorphisms were noted as an important factor in the variability of the severity of hepatitis C recurrence after liver transplantation (Rosen, 1999) and could also play a role in the variable responses to etanercept. Additional possibilities include differences in hepatitis C genotype and hold BLA genotypes.

Patients with chronic hepatitis C infection were treated with anti-tumor necrosis factor (TNF) therapy. Our clinical observations suggest that TNF plays a provocative but elusive role in the pathophysiology of hepatitis C.

MK (Patient 1) is a 59-year-old woman with a six year history of polyarticular rheumatoid arthritis. Routine laboratory examination revealed evidence of hepatitis characterized by chronic and persistent elevations of transaminases (AST, ALT) in the range of 100-200 units. Further evaluation noted a serum hepatitis C viral RNA (HCV RNA) elevated at >1,000.000 copies/ml on repeated occasions. A liver biopsy in 1997 revealed chronic active hepatitis. The patient MK did not receive any treatment for her hepatitis C infection.

In May 2000, the patient was started on Etanercept 25 mg subcutaneous injections twice weekly as treatment for her rheumatoid arthritis that was not responsive to other medications. The therapy resulted in a modest improvement of her synovitis. Remarkably, however, laboratory tests revealed normalization of her transaminases (AST, ALT) to <60 units within a few months of initiating Etanercept treatment. In addition, the HCV RNA fell to 165,000 copies/ml. These improvements persisted over four months of follow up.

Since the treatment of the patient described above, the inventors became aware of seven additional patients who had received Etanercept for the treatment of rheumatoid arthritis, who had concomitant hepatitis C infection.

Of these seven patients, described in Table 1 as patient 2-8, Patients 2 and 3 had marked (>75%) decrease in HCV RNA following Etanercept therapy. Only Patient 2 had elevated transaminases prior to therapy; these tests normalized following treatment with Etanercept. This same patient also had a marked increase of the HCV RNA following cessation of Etanercept therapy.

The remaining six patients either showed no response or had inadequate data for assessment.

A summary of the finding in the three patients whose HCV RNA responded to treatment with Etanercept is shown in Table 1. The remaining patients'results are shown in Table 2.

TABLE 1

| | HCV RNA, PCR | AST | ALT | | |
|---|---|---|---|---|---|
| | (copies/ml) | (units/L) | (units/L) | Liver Biopsy | Genotype |
| Patient 1 | | | | 1997 - chronic active | 1b |
| | | | | hepatitis 1999- chronic | |
| | | | | hepatitis with bridging | |
| | | | | fibrosis and nodule and | |
| | | | | formation, moderate | |
| | | | | steatosis | |
| 1/00 | 985,000 | 137 | 116 | | |
| 3/00 | | 100 | 102 | | |
| | >1,000,000 | 100-200 | 100-200 | | |
| 5/00 | | | | | |
| Etanercept | | | | | |
| started | | | | | |
| 7/00 | 165,000 | 177 | 124 | | |
| 9/00 | 121,000 | 48 | 53 | | |
| 11/00 | 130,000 | 156 | 171 | | |
| Etanercept | | | | | |
| discontinued | | | | | |
| Patient 2 | | | | 1993- mild nonspecific | ? |
| | | | | lymphocytic portal | |
| | | | | inflammation and minimal | |
| | | | | fatty change | |
| 11/96 | 366,000 | 58 | 39 | | |
| 12/98 | 241,000 | 68 | 37 | | |
| 12/98 | | | | | |
| Etanercept | | | | | |
| started | | | | | |
| 2/99 | 211,000 | N/A | 36 | | |
| 5/99 | 2829 | 26 | 36 | | |
| 8/99 | 798 | 38 | 36 | | |
| 9/99 | 989 | 34 | 38 | | |
| Etanercept | | | | | |
| discontinued | | | | | |
| by patient | | | | | |
| 12/99 | >1,000,000 | 37 | 40 | | |
| 2/00 | >1,000,000 | 44 | 38 | | |
| Patient 3 | | | | 1994- chronic hepatitis C | 1b |
| 12/98 | 2733 | 34 | 28 | | |
| 12/98 | | | | | |
| Etanercept | | | | | |
| started | | | | | |
| 1/99 | 27.7 | 38 | 40 | | |
| 5/99 | 37 | 37 | 35 | | |
| 6/99 | | | | | |
| Etanercept | | | | | |
| discontinued | | | | | |
| due to rash | | | | | |
| 7/00 | detected | | 38 | | |

TABLE 2

| | HCV RNA, PCR | AST | ALT | | |
|---|---|---|---|---|---|
| | (copies/ml) | (units/L) | (units/L) | Liver Biopsy | Genotype |
| Patient 4 | | | | 1998- chronic hepatitis | ? |
| | | | | C | |
| 3/98 | 443,509 | 35 | 42 | | |
| 9/99 | 14.5 | 37 | 32 | | |
| 10/99 | | | | | |
| Etanercept | | | | | |
| started | | | | | |
| 4/00 | >1,000,000 | 39 | 32 | | |
| 8/00 | detected (not | 40 | 35 | | |
| | quantitated) | | | | |
| 9/00 | | | | | |
| Etanercept | | | | | |
| discontinued | | | | | |
| Patient 5 | | | | ? | 1a |
| 5/98 | 197 | 38 | 57 | | |
| 7/99 | 851 | N/A | N/A | | |
| 10/99 | | | | | |
| Etanercept | | | | | |
| started | | | | | |
| 11/99 | 790,000 | 37 | 49 | | |
| 12/99 | >1,000,000 | 84 | 47 | | |
| 2/00 | >1,000,000 | 38 | 43 | | |
| 7/00 | 120,000 | N/A | N/A | | |
| Patient 6 | | | | 1998- chronic hepatitis | ? |
| | | | | C, moderate activity, | |
| | | | | mild to moderate | |
| | | | | fibrosis | |
| 7/98 | >1,000,000 | 256 | 97 | | |
| 9/99 | N/A | 230 | N/A | | |
| 11/99 | 131,000 | 33 | 47 | | |
| 3/00 | | | | | |
| Etanercept | | | | | |
| started | | | | | |
| 4/00 | 345,000 | 70 | 99 | | |
| 7/00 | detected (not | 71 | 130 | | |
| | quantitated) | | | | |
| 8/00 | pending | 47 | 65 | | |
| Patient 7 | | | | ? | ? |
| 3/99 | 314 | 55 | N/A | | |
| 11/99 | | | | | |
| Etanercept | | | | | |
| started | | | | | |
| 12/99 | 967,000 | 52 | 107 | | |
| 5/00 | >1,000,000 | 63 | 73 | | |
| 9/00 | >1,000,000 | 43 | 79 | | |
| Patient 8 | | | | ? | ? |
| 9/98 | <0.5 | 15 | 14 | | |
| 3/99 | | | | | |
| Etanercept | | | | | |
| started | | | | | |
| 7/99 | <2000 | 15 | 11 | | |
| 9/99 | <2000 | 14 | 10 | | |
| 5/00 | <2000 | 11 | 10 | | |

There are now some published reports supporting the effectiveness of anti-TNF-alpha therapy in hepatitis patients, for example, Tilg H., Jalan R. et A. et al. (2003) Anti-tumor factor-alpha monoclonal antibody therapy in severe alcoholic hepatitis J Hepatol 38: 419-25 and Zylberberg H., Rinaniol A C, Pol S, et al. (1999) soluble tumor necrosis factor receptors in chronic hepatitis C: A correlation with histological fibrosis and activity can be found in J Hepatol 30: 185-191.

A patient was identified with typical symptoms of rheumatoid arthritis, including polyarticular joint swelling and pain. The patient also had evidence of active hepatitis, which was characterized by chronic and persistent elevations of hepatic transaminases (AST, ALT) as well as a marked elevation of serum hepatitis C viral RNA. The patient was treated with various DMARDs and NSAIDS for a period of three years, and showed very little signs of relief from rheumatoid arthritis. The patent was then selected for treatment with Enbrel at 25 mg. twice weekly.

The rheumatoid arthritis symptoms prior to treatment included active synovitis in multiple joints (MCR, PLP, wrist, knees). The hepatitis symptoms prior to treatment included hepatitis C viral RNA of 985,000 units and abnormal liver enzymes of four years.

The patient was administered 25 mg. of Enbrel, an extracellular ligand binding protein of the human p75 TNF receptor linked to the Fc portion of human IgGl, twice weekly for five weeks. After five weeks of treatment with Enbrel, the rheumatoid arthritis showed 20-30% symptomatic improvement. The hepatitis symptoms after treatment included viral RNA of 165,000 units and a normalization of liver enzymes, including aspartate transaminase and alanine transaminase, on repeated testing after five weeks of treatment with Enbrel.

TNF neutralizing compounds were found not only to eliminate the symptoms of rheumatoid arthritis as seen in the majority of patients, but were found also to reverse the clinical symptoms associated with hepatitis, including normalization of liver enzymes and decrease in serum viral levels. These anti-inflammatory compounds demonstrate the involvement of TNF alpha in viral diseases such as hepatitis where viral infection and inflammation are closely associated. In addition to reducing inflammation, this therapy appears also to boost the immune system to reduce the viral load by 80% during a chronic infection. Therefore, anti-TNF-alpha therapy can be used for treating hepatitis-induced tumors and other viral diseases.

For treatment of hepatitis, the compounds that neutralize the activity of secreted TNF can be administered by any means and in any amount that achieves the intended propose. Amounts and regimens of a compound that neutralizes the activity of secreted TNF can be determined readily by those with ordinary skill in the art of treating diseases mediated by TNF. Generally, the compounds that neutralize the activity of secreted TNF can be administered in amounts of from about 5 mg to about 125 mg once to seven times weekly. The compounds are administered until the patient exhibits no signs of abnormal liver enzymes.

For example, administration can be by parenteral, such as by the subcutaneous, intravenous, intramuscular, intraperitoneal, transdermal, or buccal routes. Alternatively or concurrently, administration can be by the oral route, transdermally, tranmucosally, or rectally. The dosage administered depends upon the age, health, and weight of the recipient, type of treatment, frequency of the treatment, and the nature of the effect desired.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

Thus, the expressions “means to . . . ” and “means for . . . ”, or any method step language, as may be found in the specification above and/or in the claims below, followed by a functional statement, are intended to define and cover whatever structural, physical, chemical, or electrical element or structure, or whatever method step, which may now or in the future exist which carries out the recited functions, whether or not precisely equivalent to the embodiment or embodiments disclosed in the specification above, i.e., other means or steps for carrying out the same function can be used; and it is intended that such expressions be given their broadest interpretation.

What is claimed is:

1. A method for treating hepatitis comprising administering to a patient in need thereof an effective amount of a compound that neutralizes the activity of secreted TNF alpha.

2. The method according to claim 1 wherein the compound that neutralizes the activity of secreted TNF alpha is selected from the group consisting of adalimubab, indomethacin, ibuprofen, fenoprofen, sulfated disaccharides, TACE inhibitors, and atrial natriuretic peptide.

3. The method according to claim 1 wherein the activity of secreted TNF alpha is inhibited by inhibiting p75:FC.

4. The method according to claim 1 wherein the hepatitis is selected from the group consisting of hepatitis A, hepatitis B, hepatitis C, hepatitis D, and hepatitis E.

5. The method according to claim 4 wherein the hepatitis is hepatitis C.

6. The method according to claim 1 wherein the compound that neutralizes the effect of secreted TNF alpha is a humanized monoclonal antibody.

7. A method for treating hepatitis comprising administering to a patient in need thereof an effective amount of a compound that reduces viral levels in the patient.

8. The method according to claim 7 wherein the compound that reduces viral levels is a compound that neutralizes the effect of TNF alpha.

9. The method according to claim 7 wherein the hepatitis is selected from the group consisting of hepatitis A, hepatitis B, hepatitis C, hepatitis D, and hepatitis E.

10. The method according to claim 9 wherein the hepatitis is hepatitis C.

11. The method according to claim 7 wherein the compound that reduces the viral levels in the patient is a compound that inhibits p75:FC.

12. The method according to claim 7 wherein the compound that reduces the viral levels in the patient is a humanized monoclonal antibody.
